(19) United States
(12) Patent Application Publication
(10) Pub. No.: US 2008/0145813 A1
(43) Pub. Date: Jun. 19, 2008

(54) Intraoral illumination device

(76) Inventor: Bobby Crohn, Highland Mills, NY (US)

Correspondence Address: Michael E. Zall, Two Yorkshire Drive, Suffern, NY, 10901, US

(21) Appl. No.: 11/637,667

(22) Filed: Dec. 13, 2006

(51) Int. Cl. A61B 1/24; A61C 17/06

(52) U.S. Cl. 433/29; 433/138

(57) ABSTRACT

An intraoral illumination device for illuminating the interior cavity of a patient's mouth during a medical procedure. The illumination device includes a bite piece adapted to be engaged by a patient's teeth for resting the patient's jaw during the medical procedure and fixing the device within the patient's mouth. A translucent illumination member having an interior wall and an exterior wall is connected to the bite piece and optically coupled to the light source that directs light between the walls. The illumination member is a flexible arcuate member that when inserted into the mouth wraps around a rear part of the interior cavity of the patient's mouth with the interior wall facing into the mouth. The exterior wall is reflective and reflects light through the interior wall into the interior cavity of the patient's mouth. The illumination member may also have a mounting means on the exterior wall for mounting a rubber dam thereon and may further include a moisture control pad adhesively mounted to the exterior wall.

### BACKGROUND OF THE INVENTION

1. Field of the Invention

This invention relates to dental appliances, and in particular to dental appliances for illuminating the mouth of a dental patient for examination and performing operations therein.

2. Description of the Related Art

Proper illumination of a patient's mouth is essential for performing a dental examination or for performing operations therein. However, illuminating the interior of a dental patient's mouth during such examination or operation is troublesome because the patient's mouth must be illuminated through a narrow opening, i.e., the patient's mouth, and the dentist must work in close proximity to the mouth, often blocking any external light source.

Typically, the interior of the mouth is illuminated by a focused light source mounted approximately two to three feet above the dental chair within which the patient sits. The light source is configured to direct light onto and into the patient's mouth. The amount of light entering the mouth using this type of lighting is limited due to the fact that the light source is remote from the patient's mouth. Further, the dentist or oral surgeon often positions himself or his instruments between the light source and the patient's mouth in order to properly view into the patient's mouth, thus blocking light from entering the mouth.

Numerous devices have been developed through the years to overcome this problem. For example, fiber optic lighting has been incorporated into handheld dental instruments. Typically, one or two fiber optic strands extend longitudinally along the instrument and include a light outlet end configured to direct light toward the end of the instrument. However, this type of device confines the light to a limited area in the mouth and does not illuminate the entire oral cavity. Additionally, the inclusion of this type lighting in a dental instrument can interfere with the comfortable and proper use of such instrument. Additionally, the fiber optic bundles degrade over a period of time because the instrument, which includes such fiber optic lighting, is autoclaved numerous times in order to sterilize the instrument.

Other devices have been designed for illuminating a patient's teeth. However, most of these devices are not optimum because they do not provide adequate illumination of the patient's teeth, and/or they interfere with the use of other dental instruments during the examination and/or operation.

Examples of known intraoral lighting devices are shown in the following US Patents and publications:

U.S. Pat. No. 6,338,627 and U.S. Pat. No. 6,022,214 to Hirsch et al—These patents are related to the commercially available Isolite Disposable Mouthpiece sold by Isolite Systems (Santa Barbara, Calif.; www.isolitesystem.com).

| | |
|---|---|
| 6,080,105 to Spears. | 4,807,599 to Robinson, et al. |
| 6,332,776 to Martin, et al. | 4,592,344 to Scheer |
| 6,176,824 to Davis | 4,589,846 to Annoni |
| 6,739,744 to Williams, et al. | 4,344,419 to Burgin |
| 6,591,049 to Williams, et al. | 2,528,458 to Stone |
| 6,059,723 to Davis | D458,680 to Engler |
| 5,656,014 to Rooney, et al. | D422,081 to Wolff |
| 5,355,870 to Lacy | |
| 5,318,009 to Robinson | |

### SUMMARY OF INVENTION

The present invention is directed to an intraoral illumination device for illuminating the interior cavity of a patient's mouth during a medical procedure. The illumination device includes a bite piece adapted to be engaged by a patient's teeth for resting the patient's jaw during the medical procedure and fixing the device within the patient's mouth. A translucent illumination member having an interior wall and an exterior wall is connected to the bite piece and optically coupled to the light source that directs light between the walls. The illumination member is a flexible arcuate member that when inserted into the mouth wraps around a rear part of the interior cavity of the patient's mouth with the interior wall facing into the mouth. The exterior wall is reflective and reflects light through the interior wall into the interior cavity of the patient's mouth. The illumination member may also have a mounting means on the exterior wall for mounting a rubber dam thereon and may further comprise a moisture control pad adhesively mounted to the exterior wall.

Further features and advantages of the invention will become apparent from a consideration of the following description and the appended claims when taken in connection with the accompanying drawings.

### BRIEF DESCRIPTION OF THE DRAWINGS

For a more complete understanding of the present invention, reference may be had to the following description of the exemplary embodiments of the present invention considered in connection with the accompanying drawings, of which:

FIG. 1 is a perspective view of the intraoral illumination device of this invention with the light source uncoupled therefrom;

FIG. 2 is a side elevational view of the intraoral illumination device of FIG. 1 in a patient's mouth;

FIG. 3 is an exploded top plan view the intraoral illumination device of FIG. 1; and

FIG. 4 is a cross-sectional view of the intraoral illumination device of this invention taken along line 4-4 of FIG. 2.

### DETAILED DESCRIPTION OF THE INVENTION

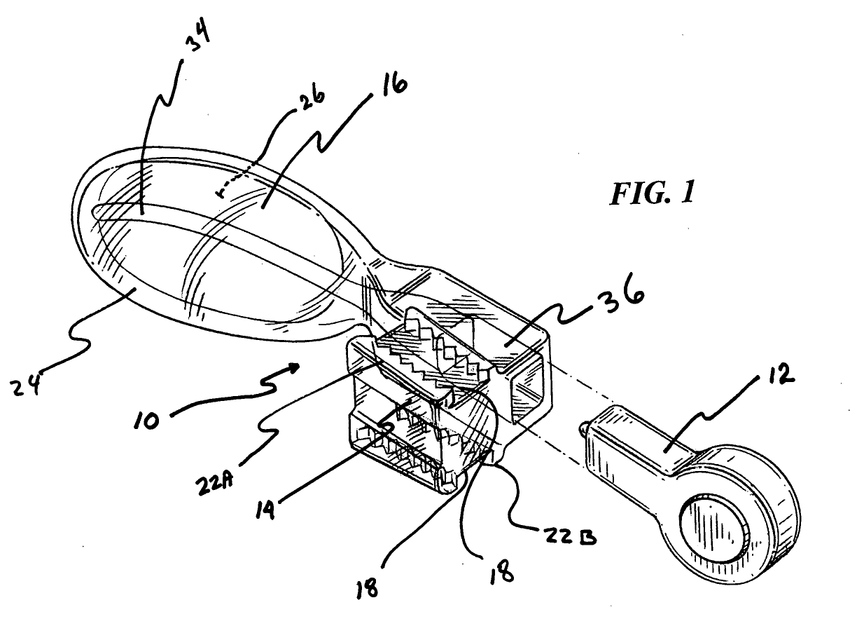

Referring to FIG. 1-4, the intraoral illumination device 10 of this invention is shown. The device 10 includes a light source 12, a bite piece 14 and a translucent illumination member 16.

The bite piece 14 is coupled to a light source 12 which can be the portable light shown FIGS. 1-4 or an extraoral fiber optic bundle light source (not shown) for illuminating the translucent illumination member 16.

The bite piece 14 and the translucent illumination member 16 are preferably contiguous and molded from a translucent, biologically acceptable polymer. The bite piece 14 and a translucent illumination member 16 may be sterilizable for reuse or disposable. Preferably, the bite piece 14 and translucent illumination member 16 are made of a single molded piece of soft, flexible, biocompatible material such as Pebax® (Arkema, Inc.), Santoprene rubber, or a molded vinyl material. However, it will be readily understood by those skilled in the art that other soft, flexible materials could be used.

The bite piece 14 and the translucent illumination member 16 may be produced by a gas-assist injection molding process or other molding processes such as an injection molding process. Optionally, the bite piece 14 and a translucent illumination member 16 may be separate interlocking pieces that can be easily assembled.

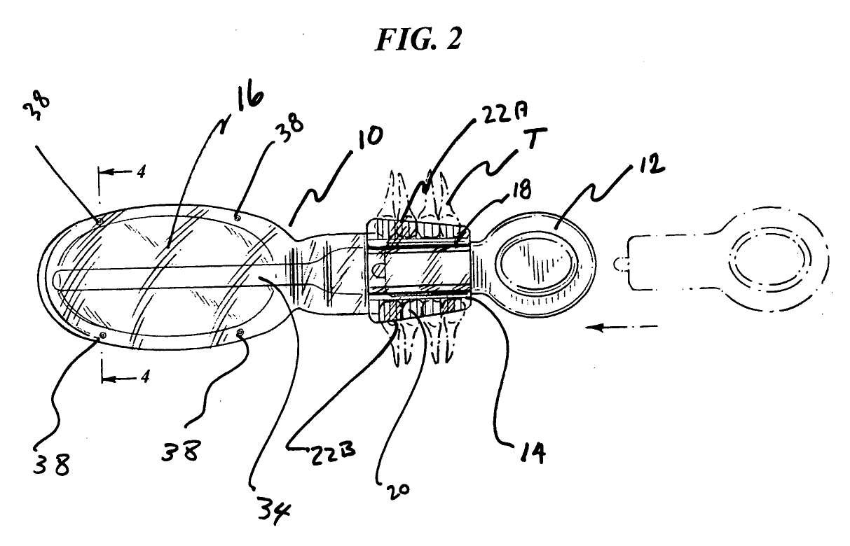

Referring to FIGS. 1 and 2, the bite piece 14 is adapted to be engaged by a patient's teeth to hold the device 10 within the patient's mouth. The bite piece 14 is made of a single piece of biocompatible, sterilizable material such as rubber. Referring to FIG. 1, the bite piece 14 includes a channel 18 and ribs 20 along opposite faces 22A, 22B of the bite block 14. The channel 18 and ribs 20 help to prevent the bite block 14 from slipping when inserted in the mouth between the molar and bicuspid teeth T of the patient. When held or engaged between the patient's teeth, the bite block 14 functions to hold the illumination member 16 in a rear, central part of the interior cavity of the patient's mouth so that light can be transmitted outward therefrom for illuminating the interior cavity.

Referring to FIGS. 1-4, the translucent illumination member 16 includes an interior wall 24 and an exterior wall 26 connected to the bite piece 14. It is optically coupled to the light source 12 which directs light between the walls 24, 26. The illumination member 16 is a flexible arcuate member that when inserted into the mouth wraps around a rear part of the interior cavity of the patient's mouth. The interior wall 24 of the illumination member 16 faces into the mouth and the exterior wall 26 of the illumination member 16 is juxtaposed against the interior surface (cheek) of the mouth.

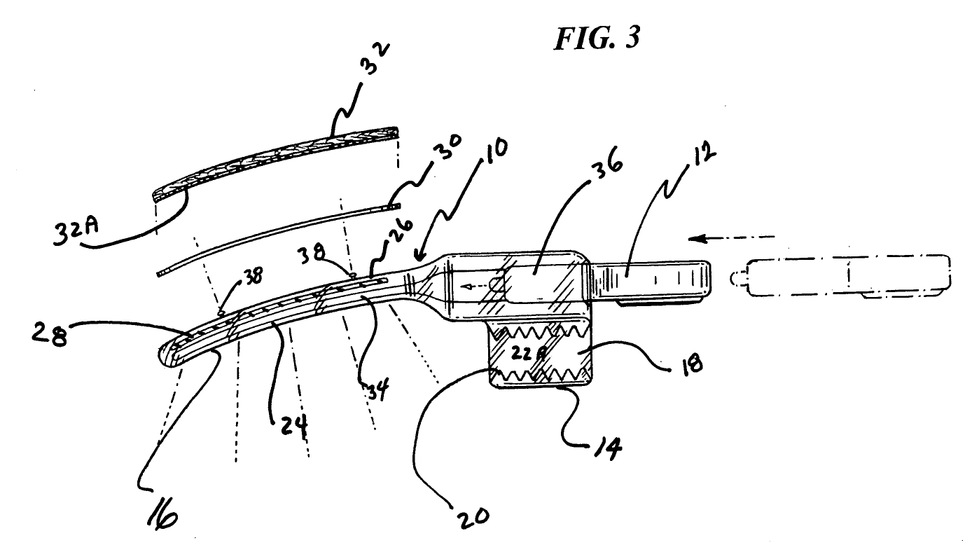

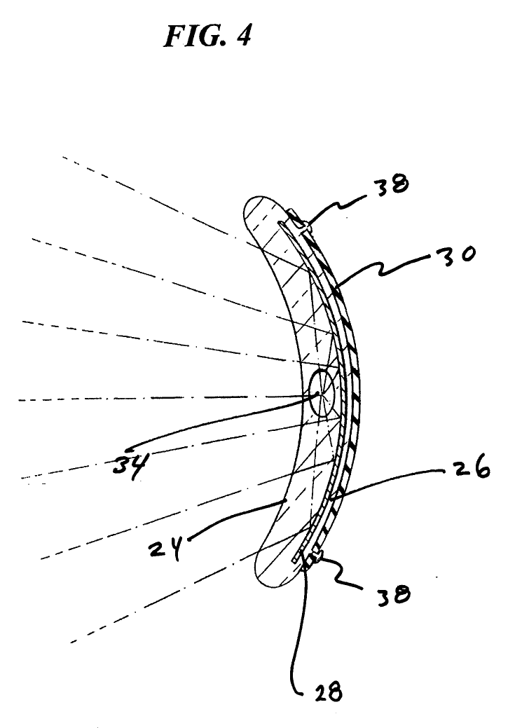

The illumination member 16 may be made of a single, injection-molded piece of light-dispersive, biocompatible, sterilizable material. The member 16 may be made of a rigid material such as acrylic or a flexible material such as a molded flexible urethane. However, it will be readily understood by those skilled in the art that other clear, flexible or rigid materials may be used. Referring to FIGS. 3 and 4, the illumination member 16 has an arcuate, semi-circular shape.

Still referring to FIGS. 3 and 4, the illumination member 16 further includes in close proximity to the exterior wall 26 a reflective surface 28 that reflects the light emanating from the light source 12 through the illuminating member 16 toward interior wall 24 and into the interior cavity of the patients mouth. The reflective surface 28 that is in close proximity to the exterior wall 24 may be created by numerous means. For example, as shown in FIGS. 3 and 4, a reflective sheet 28 may be embedded in close proximity the exterior wall 26. Optionally, such reflective sheet may be mounted to the exterior wall 26 or formed by coating the exterior wall. However created, the function of such reflective surface is to prevent the escape of light through the exterior wall and concentrate and direct the light into the interior of the mouth to enhance visibility therein.

Referring to FIGS. 2, 3 and 4, the illumination member 16 may additionally have mounting means, for example nubs 38, on the exterior wall 16 for mounting a rubber dam 30 thereon. Generally, a rubber dam 30 is a flat thin sheet of latex or non-latex that is held in place by, for example a clamp and frame, and in this case by nubs 38. The dam 30 is perforated (not shown) to allow the teeth that will be worked on to protrude through the perforations in the sheet while all the other teeth are covered and protected by the rubber dam.

Referring to FIG. 3, the illumination member 16 may additionally have a moisture control pad 32, e.g., dry angle, mounted by an adhesive layer 32a to the exterior wall 26 of the illumination member 16. This moisture control pad 32 will prevent saliva buildup caused, for example, by the Stensons duct which is next to the upper 1^{st} molars. During dental examination and/or operation, a number of fluids, e.g., saliva from the parotid gland, blood, water from the dental equipment, are produced in the patient's mouth. It is important to remove these fluids for the comfort of the patient, to prevent fluids and material from being aspirated into the throat or lungs of the patient, and to assist the health care provider in observing and/or operating within the patient's mouth. The moisture control pads 32 assists in removing fluids from the mouth.

The illumination member 16 optically communicates with the light source 12 through a stem or channel 34 that passes between the interior and exterior walls 24, 26 of the illumination member 16. The illumination member 16 includes a light carrier 36 adapted to be directly connected to a commercially available illumination source 12 such as those found in most dentist offices or adapted to be connected to such sources through a connector or transition mechanism (not shown).

Referring to FIGS. 3 and 4, in use, light shines inward from the walls 24, 26 to illuminate the patient's mouth. The generally arcuate shape of the illumination member 16 and the dispersional qualities of reflective surface 28 spreads the total area of illumination. Illuminating the mouth from the central, rear part of the intraoral cavity eliminates shadows caused by a single-point light source, and shadows caused by the health care provider or equipment used by the health care provider.

The intraoral illumination device 10 may also come in different sizes and configurations and to accommodate different mouth sizes and shapes.

Although this invention has been described in connection with illuminating, isolating, and removing fluids from a patient's mouth for dentistry, it will be readily understood by those skilled in the art how the present invention may have other mouth-related applications where illumination in the mouth is required other than dentistry such as, but not by way of limitation, oral surgery.

The foregoing constitutes a description of specific embodiments showing how the invention may be applied and put into use. These embodiments are only exemplary. The invention in its broadest, and more specific aspects, is further described and defined in the claims which now follow.

These claims, and the language used therein, are to be understood in terms of the variants of the invention which have been described. They are not to be restricted to such variants, but are to be read as covering the full scope of the invention as is implicit within the invention and the disclosure.

1. An intraoral illumination device for illuminating the interior cavity of a patient's mouth during a medical procedure, the intraoral illumination device for use with a light source, comprising:
a bite piece adapted to be engaged by a patient's teeth to hold the device within the patient's mouth;
a translucent illumination member having an interior wall and an exterior wall substantially parallel to the interior wall, the illumination member connected to said bite piece and optically coupled to the light source that directs light between the walls,
wherein the illumination member is a flexible arcuate member whereby when inserted into the mouth the interior and exterior walls wrap around a rear part of the interior cavity of the patient's mouth with the interior wall facing into the mouth,
wherein the exterior wall is a reflective arcuate surface that prevents escape of light through the exterior wall and reflects and directs light through the interior wall into the interior cavity of the patients mouth to enhance visibility therein.

2. (canceled)

3. The device of claim 1, wherein the illumination member has mounting means on the exterior wall for mounting a rubber dam thereon.

4. The device of claim 1, further comprising a moisture control pad removably mounted to the exterior wall.

5. The device of claim 1, further comprising a moisture control pad adhesively mounted to the exterior wall.

6. The device of claim 1, wherein the illumination member is fixedly connected to the bite piece so as not to move relative thereto.

7. The device of claim 1, wherein the device is a single-use, disposable device.

8. An intraoral illumination device for illuminating the interior cavity of a patient's mouth during a medical procedure, the intraoral illumination device for use with a light source, comprising:
a bite piece adapted to be engaged by a patient's teeth to hold the device within the patient's mouth;
a translucent illumination member having an interior wall and an exterior wall substantially parallel to the interior wall, the illumination member connected to said bite piece and optically coupled to the light source that directs light between the walls,
wherein the illumination member is a flexible arcuate member whereby when inserted into the mouth the interior and exterior walls wrap around a rear part of the interior cavity of the patient's mouth with the interior wall facing into the mouth,
wherein the exterior wall is a reflective arcuate surface that prevents escape of light through the exterior wall and reflects and directs light through the interior wall into the interior cavity of the patients mouth to enhance visibility therein.
wherein the exterior wall has thereon a plurality of nubs for mounting a rubber dam thereon.
